(19) United States
(12) Patent Application Publication
(10) Pub. No.: US 2012/0226247 A1
(43) Pub. Date: Sep. 6, 2012

(54) WOUND TREATMENT ARRANGEMENT AND SUCTION CONNECTION FOR A WOUND TREATMENT ARRANGEMENT

(75) Inventors: Federico Danei, Vienna (AT); Georg Wagner, Vienna (AT); Christian Rohrer, Linz (AT); Erik Steinlechner, Baden (AT)

(73) Assignee: LOHMANN & RAUSCHER GMBH, Schonau an der Triesting (AT)

(21) Appl. No.: 13/410,160

(22) Filed: Mar. 1, 2012

(30) Foreign Application Priority Data
Mar. 2, 2011 (EP) 11 001 737.3

(51) Int. Cl. A61M 1/00

(52) U.S. Cl. 604/319

(57) ABSTRACT

The invention relates to a wound treatment arrangement having a filling material which is designed for filling the wound, and a suction connection which is designed for suctioning wound exudate out of the filler material, and which is to be arranged on the side facing away from the wound bed, outside of the filler material, with a drainage layer arranged between the filler material and the suction connection, to introduce the exudate to be suctioned out of the filler material, into at least one suction opening of the suction connection.

### CROSS REFERENCE TO RELATED APPLICATION

The present application claims international priority under 35 U.S.C. §119 to co-pending European Patent Application No. 11 001 737.3, filed Mar. 2, 2011, entitled “Wundversorgungsanordnung and Sauganschluss Fur Eine Wundversorgungsanordnung,” the entire content and disclosure of which is hereby incorporated by reference in its entirety.

### TECHNICAL FIELD

The invention relates to a wound treatment arrangement with a filler material designed for filling the wound, and a suction connection designed for suctioning wound exudate out of the filler material, and to be arranged on the side facing away from the wound bed, outside of the filler material, as well as to a suction connection for such a wound treatment arrangement.

### BACKGROUND

Wound treatment arrangements are used particularly in the context of the so-called vacuum therapy. It has been found that the healing of chronic wounds in particular can be promoted by the application of a negative pressure to said wounds. In the process, it has also been found to be advantageous if the wound is covered or filled with an open-pored foam or gauze as filler material, and a suction connection is applied to the side of the filler material, which is facing away from the wound bed, and connected to a suction device designed for the generation of a negative pressure. For this purpose, the suction connection can be provided, for example, with a tube which is connectable, on the one hand to a connection device of the suction connection, which is designed, for example, in the form of a pipe connector, and, on the other hand, to the suction device. In addition, a cover device, designed, for example, as a film-like material, can be provided, which is applied in an air-tight manner to the skin surface adjacent to the wound, and which forms a space which is closed off in an air-tight manner and contains the filler material, and which is connected via the suction connection to the suction device, to generate a negative pressure within said space.

Wound treatment arrangements that can be used in the context of vacuum therapy are described, for example, in EP 0 620 720 B1. The disclosure content of said document is hereby included by explicit reference in this description, with regard to the details on the foam forming agent which can be used in the context of the vacuum therapy, and on the suction devices.

In DE 10 2009 019 646 A1, a contact layer to be inserted between the filler material and the wound bed, for the purpose of improving the exudate management, is described, which layer forms a drainage space between the filler material and the wound bed. The disclosure content of said patent is included explicitly in this description, with regard to the details of the contact layer forming the drainage space, or of the wound cover.

Suction connections which are usable in the context of vacuum therapy, and which can be connected via a tube to a suction device, are described, for example, in WO 03/073970 A1, WO 2008/014358 A2, and WO 2009/124548 A1. A suction connection referred to as a suction head, with protrusions functioning as flow guides in the area of the delimitation surface of the suction connection, which faces the wound, is described in EP 1 018 967 B1. Furthermore, a suction connection is indicated in EP 1 088 569 B1, which has an application surface which is to be applied on the filler material, and which is in the shape of a disk-shaped cup. In a suction connection described in WO 2010/008167 A2, channels are formed, which are delimited by bars on the delimitation surface facing the filler material, and the purpose of which is to guide the wound exudate in the direction towards a suction opening. Finally, in EP 2 098 257 A1, a suction connection is described for use in vacuum therapy, through which a plurality of suction channels passes, wherein a central suction channel is surrounded by a plurality of suction channels of smaller cross section, which open into the central suction channel.

In DE 20 2010 005 872.4, a suction connection is indicated, wherein a suction opening facing the filler material is surrounded by an annular protrusion.

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation of the accompanying drawings.

FIG. 1 shows a suction connection according to the invention according to a first embodiment of the invention,

FIG. 2 shows a suction connection according to the invention according to a second embodiment of the invention,

FIG. 3 shows a wound treatment arrangement according to the invention, according to a first embodiment of the invention,

FIG. 4 shows a wound treatment arrangement according to the invention, according to a second embodiment of the invention,

FIG. 5 shows a suction connection according to a third embodiment of the invention,

FIG. 6 shows a wound treatment arrangement designed with the suction connection according to FIG. 5,

FIG. 7 shows a suction connection according to a fourth embodiment of the invention,

FIG. 8 shows a wound treatment arrangement with a suction connection according to FIG. 7,

FIG. 9 shows a suction connection according to a fifth embodiment of the invention,

FIG. 10 shows a wound treatment arrangement with a suction connection according to FIG. 9, and

FIG. 11 shows a suction connection according to a sixth embodiment of the invention.

### DETAILED DESCRIPTION OF EMBODIMENTS

It has been found that, when using the known suction connections, problems are observed in many cases during the removal of wound exudate.

In view of said problems in the prior art, the invention is based on the problem of providing a wound treatment arrangement by means of which a satisfactory removal of wound secretion out of the wound area is reliably possible.

According to the invention, this problem is solved by a variant of the known wound treatment arrangements, which is substantially characterized in that, between the filler material and the suction connection, a drainage layer is provided, for introducing the exudate to be suctioned out of the filler material into at least one suction opening of the suction connection.

Here, the invention is based on the knowledge that, in the known suction connections, protrusions or bars provided on the side facing the filler material prevent the removal of wound exudate out of the filler material, due to the fact that they become embedded in the filler material, due to the action of the differential pressure between the outer delimitation surface of the suction connection, which faces away from the filler material, and the delimitation surface of the suction connection, which faces the filler material, and in this manner they compromise the properties of the filler material designed, for example, as an open-pored foam or gauze, precisely in the area of the suction connection, properties which otherwise promote the removal of the wound exudate.

In suction connections according to EP 2 098 257 A1, the problem observed is that the suction channels of small cross section can easily become clogged, if there is coagulation of wound exudate in the form of clots.

Said problems are solved by using the wound treatment arrangements according to the invention, because the application surface of the suction connection, which faces the wound bed, is no longer applied directly on the filler material, rather it is applied on the drainage layer, through which the wound exudate taken up by the filler material is led to the suction opening.

In the wound treatment arrangement according to the invention, channel-forming protrusions are not required on the delimitation surface of the suction connection, which faces the filler material, and consequently no enlargement of the suction surface, in the form of a disk-like cup, is required. Instead, it is sufficient if the delimitation surface of the suction connection, which faces the filler material, has a flange-like flat design, and is perforated by a suction opening, because the introduction of the wound exudate from the filler material is not produced by a special feature of the connection, but by the drainage layer arranged between the connection and the filler material. If the application surface of the suction connection, which is to be applied against the drainage layer, has a flat design, i.e., without protrusion, in the form of bars that delimit channels, or in the form of annular protrusions, as in the disk-like cups according to EP 1 088 569 B1, the pushing in of protruding structures into the drainage layer is prevented, and thus a reliable operation of the drainage layer is ensured.

In a manner similar to conventional suction connections, on the side of the suction connection according to the invention, which faces away from the application surface, a connection device, for example, a pipe connector, can be provided, by means of which an air-tight connection can be established between the suction opening which faces the filler material, and a suction tube which is arranged on the side facing away from the filler material, outside of the filler material.

In terms of a particularly simple use of wound treatment arrangements according to the invention, it has been found to be advantageous if the drainage layer is attached on the application surface, particularly glued, welded on the application surface, clamped and/or sewn on the application surface. The entire arrangement, consisting of the suction connection and the drainage layer, can then be positioned as a whole at the desired location on the filler material.

It was found surprisingly that, in vacuum therapy, a continuous air entry into the wound area, which occurs simultaneously with the generation of the negative pressure, further improves the healing process. Therefore, a wound treatment arrangement according to a particularly preferred embodiment of the invention comprises a suction connection which also comprises, besides a suction opening used for suctioning the exudate, a ventilation opening which is arranged particularly in the flange-like application area of the suction connection, and used to ventilate the wound. A continuous air entry into the wound area, which is made possible by the ventilation opening, causes a controlled and continuous pressure decrease at the time of the connection of a suction device to the suction opening. As a result, the removal of the exudate can be further improved. The pump used to suction the exudate then generates a higher throughflow and an improved suction action. It enters into action more often, and it suctions more.

To prevent contamination of the wound, the ventilation opening is advantageously associated with an antibacterial filter which can be arranged in the ventilation opening, or cover the ventilation opening. The filter is advantageously hydrophobic, and, to achieve the desired filtering action, it has a pore size of 0.001, particularly 0.005, preferably 0.02, particularly preferably 0.1-5 μm. In the case of a pore size of less than 0.001 μm, the desired ventilation is affected. In the case of a pore size of more than 5 μm, the antibacterial effect is still barely achieved. The material of the filter can contain particularly polytetrafluoroethylene. By selecting the pore size of the filter, the air throughflow in the wound area is also influenced, wherein a decrease in the size of the pores leads to a decrease of the entry of air.

Additionally or alternatively to the ventilation opening, the wound treatment arrangement can also comprise a multilumen tube facing the suction opening, which may in particular contain three lumens, of which only one is used for suctioning the exudate, another for the controlled air supply, and a third for measuring the pressure immediately at the wound. By using the multilumen tube, the suction action can be improved, without the suction connection requiring any changes in construction. However, if an additional ventilation opening is used, the suction action is improved even more. A further improvement can be achieved, if the pipe connector used as connection device comprises a number of lumens corresponding to the multilumen tube.

In terms of an optimal wound treatment, without the removal of a wound treatment arrangement according to the invention, it has been found to be advantageous if the suction connection also comprises, in addition to the suction opening, a supply opening which is designed to supply a wound treatment product, and which preferably passes completely through the application area, wherein, in a particularly preferred embodiment of the invention, said supply opening can be associated on the side of the suction connection, which is turned away from the application surface, with an additional connection device, such as, for example, an additional pipe connector, for establishing a connection between the supply opening and a feed tube. Through the supply opening, it is possible to introduce, for example, a rinsing solution, optionally with drugs, disinfectants and the like, into the wound area. The supply opening, just like the suction removal opening, can be covered by the drainage layer. However, in the context of the invention, it is particularly preferred if the opening is not covered by the drainage layer, in order to improve the diffusion of the rinsing solution into the wound area.

To further improve the exudate management, the wound treatment arrangement according to the invention can comprise a contact layer, which is arranged between the wound bed and the filler material, and which produces drainage on the wound side.

In the context of the invention, it has been found to be particularly advantageous if the drainage layer and/or the contact layer, in accordance with the wound covers according to DE 10 2009 019 646 A1, comprises two approximately mutually parallel track-shaped elements, between which a drainage space is formed, the depth of which ensures, in a depth direction extending approximately perpendicularly to the track-shaped elements, a capillary action on the exudates taken up in the drainage space. The depths of the drainage space for this purpose can be 5 mm or less, and 0.5 mm or more. Here, each one of the track-shaped elements advantageously comprises an opening that allows the passage of body fluid into the drainage space, wherein at least one opening is formed by a channel which extends, starting from one of the track-shaped elements, in the direction towards the opposite inner delimitation surface of the other track-shaped element, and which opens into the drainage space, the channel wall of said channel being designed to form a single piece with a track-shaped element, particularly by perforation of the track-shaped element.

In terms of a particularly pronounced capillary action, it has been found to be advantageous if the cross-sectional surface of the channel decreases in a plane extending perpendicularly to the depth direction, starting from the track-shaped element, in the direction towards the opposite other delimitation surface, in particular to obtain a capillary action which favors the entry of body fluid into the drainage space. Here, at least one track-shaped element can comprise a plurality of openings arranged preferably in a grid pattern, wherein the separation between adjacent openings is 15 mm or less, preferably 5 mm or less, particularly 3 mm or less, and the apertures of the openings arranged in a track-shaped element are arranged in a projection along the depth direction between the apertures of the openings arranged in the other track-shaped element, wherein at least one channel extends in the depth direction over 50% or more of the entire depth of the drainage space.

The channel wall of the channels forming the opening is designed at least in sections in the shape of an arc, in a cross-sectional plane extending parallel to the depth direction, and it transitions continuously into the delimitation surface of the track-shaped element. Additional characteristics of contact and/or drainage layers that can be used according to the invention are indicated in DE 10 2009 019 646 A1, the disclosure content of which is hereby included by explicit reference in this description.

As can be seen from the explanation of known wound treatment arrangements, the wound treatment arrangement according to the invention, for use in vacuum therapy, also comprises advantageously a cover device, for example, a cover film, which is applied in an air-tight manner to the skin surrounding the wound, and which is used to form a space which is sealed off in an air-tight manner and contains the filler material.

If the drainage layer covers the application surface of the suction connection only partially, wherein preferably at least the suction opening and the filter are covered, and an attachment area surrounding the drainage layer is provided on the application surface of the suction connection, the suction connection can be glued with said attachment area to the cover device. For this purpose, the attachment area of the suction connection can be provided with an appropriate adhesive. Alternatively, it is also possible, for example, to use a double-sided adhesive tape, instead of adhesive. The adhesive (for example, acrylate, silicone, polyurethane) can here be applied partially (for example, in rings), porously or over the entire surface. The adhesive tape as well can be applied partially (for example, in rings) or over the entire surface. Moreover, the double-sided adhesive tape can be coated on both sides with the same glue (for example, acrylate, silicone, polyurethane), or the two sides can be coated with two different adhesives (particularly on the top side, in contact with the application surface 12, with silicone adhesive, and on the bottom side, with acrylate adhesive). Both the adhesive and the adhesive tape can be provided with a detachable protection layer.

If the drainage layer covers the application surface of the suction connection completely, the cover device, which may have a film-like design, for example, can be glued to an attachment area of the suction connection, which is facing away from the application surface, and which surrounds the connection device of the suction device, said connection device being designed, for example, in the form of a pipe connector.

As in known wound treatment arrangements for use in vacuum therapy, the filler material of wound treatment arrangements according to the invention can present an open-pored foam or gauze.

A suction connection according to the invention, which is designed for use in a wound treatment arrangement according to the invention, is characterized substantially in that it presents a substantially flat application surface, by means of which it can be applied to the drainage layer, so that the penetration of protrusions into the drainage layer and/or the filler material is prevented reliably. Here, the application surface is penetrated by a suction opening which is connected to a connection device in the form of a pipe connector on the side of the connection, which faces away from the wound, to ensure in this manner an air-tight connection of a suction tube. Via the suction tube, with the help of a suction device, exudate can be removed by suction via the suction opening in the application surface, and a negative pressure can be generated in the space filled with the filler material, to promote wound healing in this manner.

As already explained above, the drainage layer of a wound treatment arrangement according to the invention can be attached, particularly glued, welded, to the application surface of the suction connection according to the invention, clamped and/or sewn on the suction connection.

According to a particularly preferred embodiment of the invention, the suction connection presents an application area which is penetrated by a suction opening, and in particular flange-like, and which, in addition, is also completely perforated by a ventilation opening and/or a supply opening for supplying a wound treatment product, wherein the supply opening can be associated with an additional connection device designed in particular like a pipe connector, to establish a connection between the supply opening and a supply tube.

The invention is explained below in reference to the drawing, to which explicit reference is made with regard to all details essential to the invention but not emphasized in greater detail in the description.

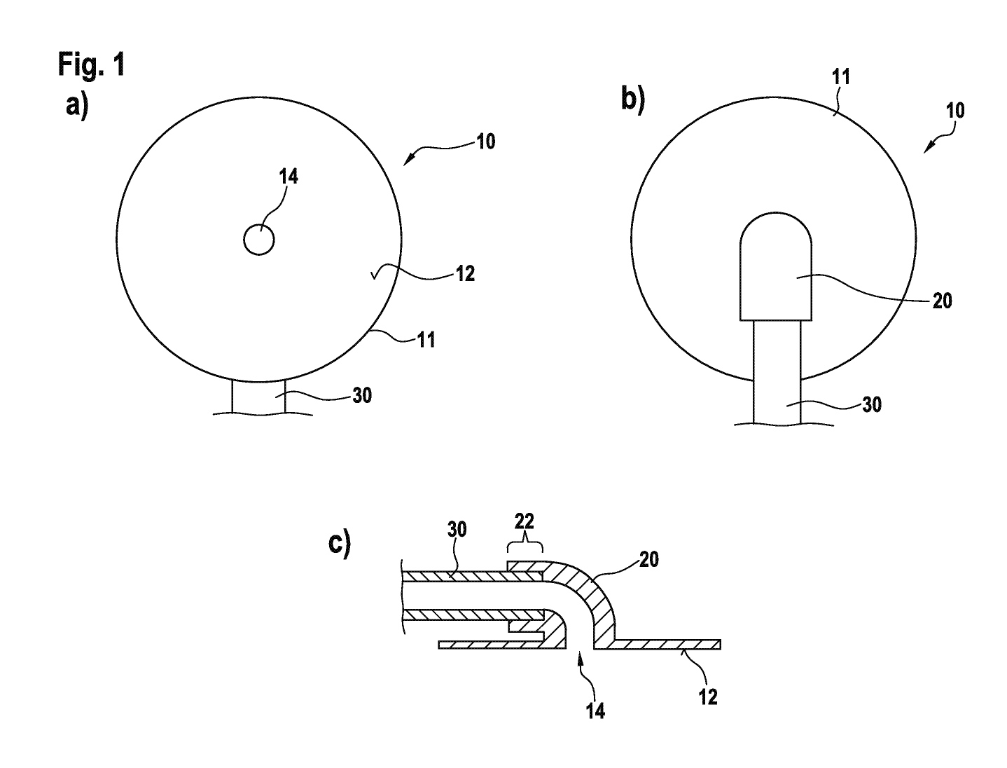

FIG. 1a) shows a view from below of a suction connection 10 according to the invention; FIG. 1b) shows a view from top of the suction connection 10 according to FIG. 1a); and FIG. 1c) shows a cross-sectional representation of a suction connection 10 according to the invention.

The suction connection 10 represented in FIG. 1 presents a flange-like application area 11 with a circular disk shaped, flat application surface 12 without protrusions. The application surface 12 is completely perforated by a suction opening 14. Here, the suction opening 14 which is also circular is arranged in the center of the application surface 12. The suction opening 14 opens into a connection device 20, which is designed like a pipe connector, and which produces a deflection by 90° of the exudate flow which is oriented perpendicularly to the application surface 12, so that the flow is oriented approximately parallel to the application surface 12, after the deflection. The pipe connector 20 presents, at its end facing away from the suction opening 14, a connection area 22 with enlarged inner diameter, into which the suction tube 30 can be introduced in an air-tight manner. Due to the deflection of the suction flow with the help of the pipe connector 20, an orientation of the suction tube 30 in a direction parallel to the application surface 12 is made possible. As a result, a smooth application of the suction connection 10, and of the suction tube 30 connected to it, to the patient's body is possible.

In the embodiment represented in FIG. 1c), the suction tube 30 is dimensioned in such a manner that its inner delimitation surface is in alignment with an inner surface area of the pipe connector 20, which abuts against the connection area 22, to minimize the flow resistance for the wound exudate.

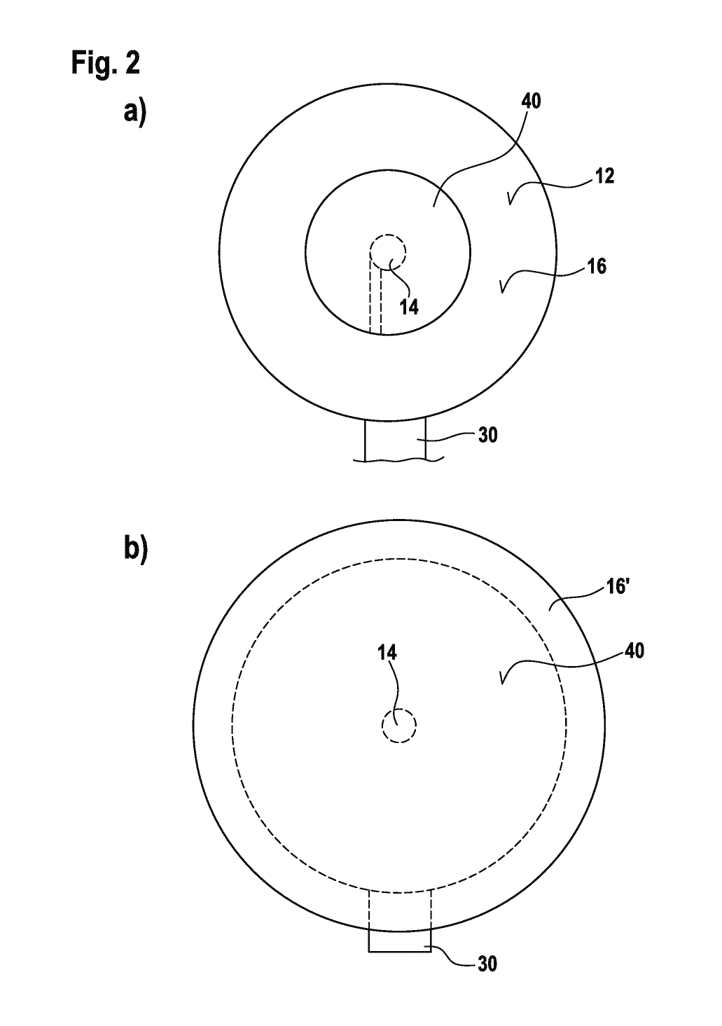

In the embodiment according to FIG. 2a), a central area of the application surface 12, which contains the suction opening 14, is covered by a drainage layer 40 designed to introduce wound exudate into the suction opening 14. The drainage layer 40 is glued to the application surface 12, and it is surrounded by an attachment area 16 of the application surface 12 in the form of a ring.

In the embodiment according to FIG. 2b), the application surface 12 is covered completely by the drainage layer 40. On the delimitation surface of the suction connection 10, which is facing away from the application surface 12, an attachment area 16′ which surrounds the pipe connector 20 is provided, and which, like the attachment area 16, can be provided with an adhesive layer which, before the use of the suction connection 10 in a wound treatment arrangement according to the invention, can be covered by a detachable protection layer.

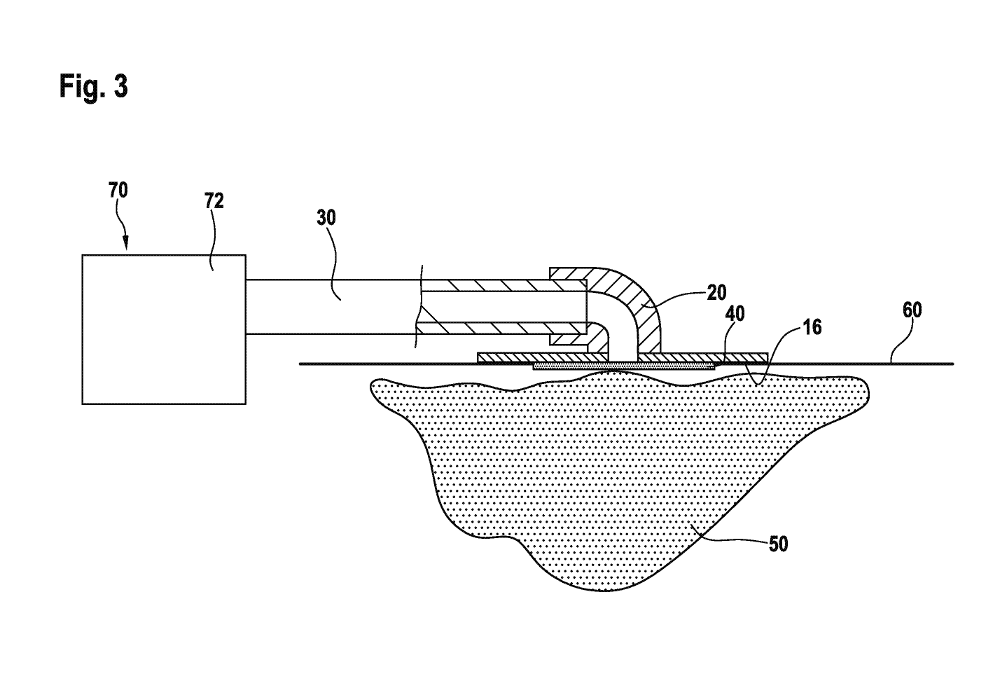

FIG. 3 shows the use of a suction connection 10 according to FIG. 2a) in a wound treatment arrangement according to the invention, which comprises a filler material 50 which fills a wound, wherein a cover film 60 is glued to the skin surrounding the wound. The cover film 60 presents a central opening which is completely perforated by the drainage layer 40 glued to the application surface 12, so that the wound exudate can be introduced, from the filler material 50, through the drainage layer 40, into the pipe connector 20, and suctioned via the suction tube 30 with a suction device 70 into a collecting vessel 72.

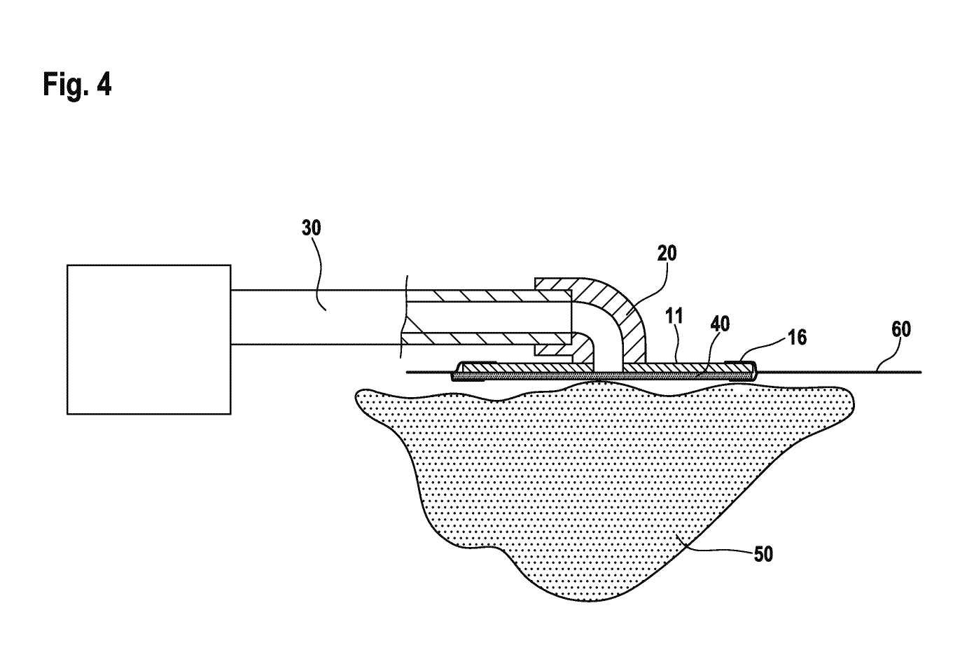

The embodiment according to FIG. 4 differs from the embodiment according to FIG. 3 in that the suction connection 10 according to FIG. 2b) is used, and the cover film 60 is glued to the delimitation surface facing away from the filler material 50, and to the attachment area 16 which surrounds the pipe connector 20 in the form of a ring. In addition, a marginal area of the drainage layer 40, which surrounds the suction opening 14, can be glued on an attachment flap of the cover film 60, so that a margin which comprises the drainage layer 40 and the suction connection 10 is accommodated in a pocket of the cover film 60. As a result, a particularly air-tight connection of the suction connection 10 to the cover film 60 is achieved.

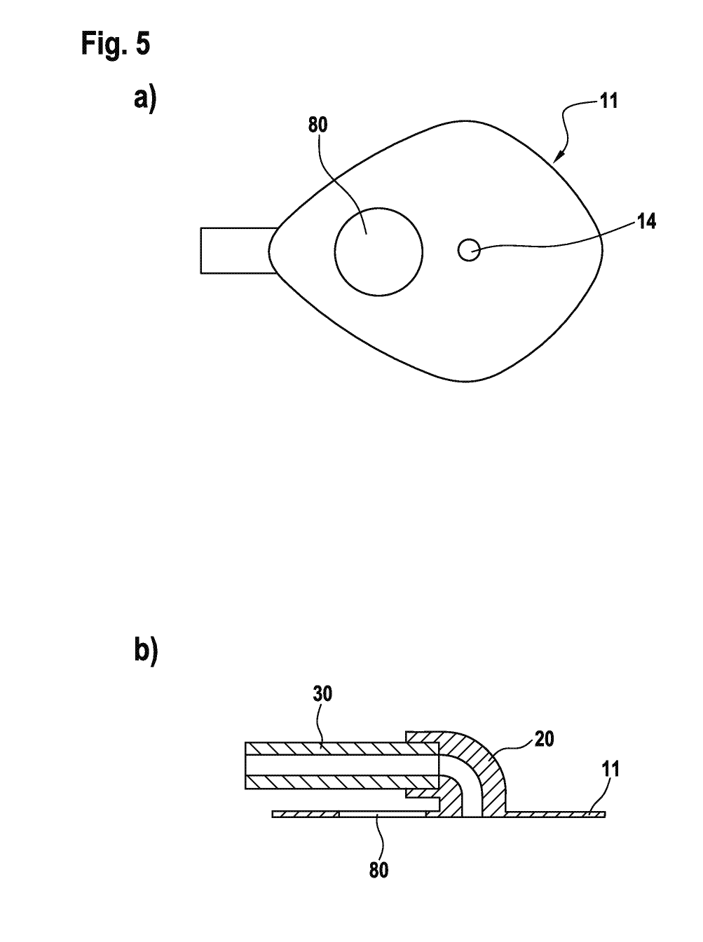

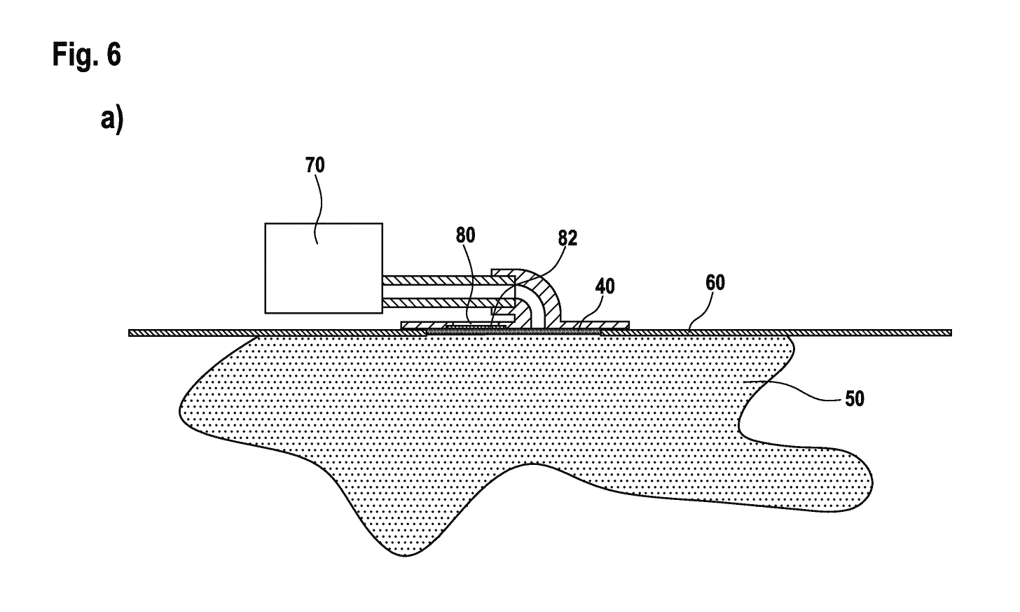

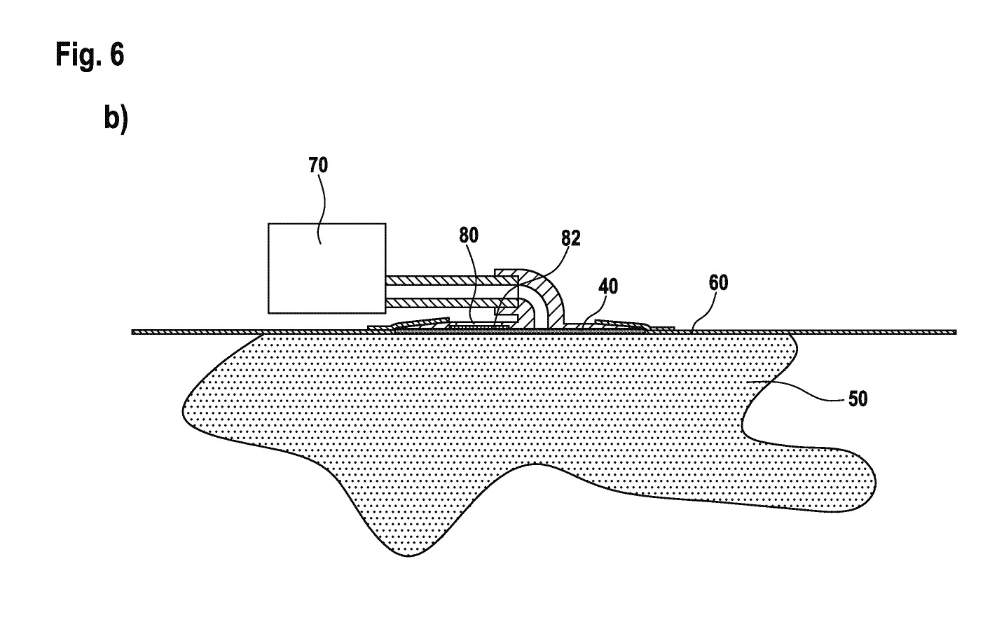

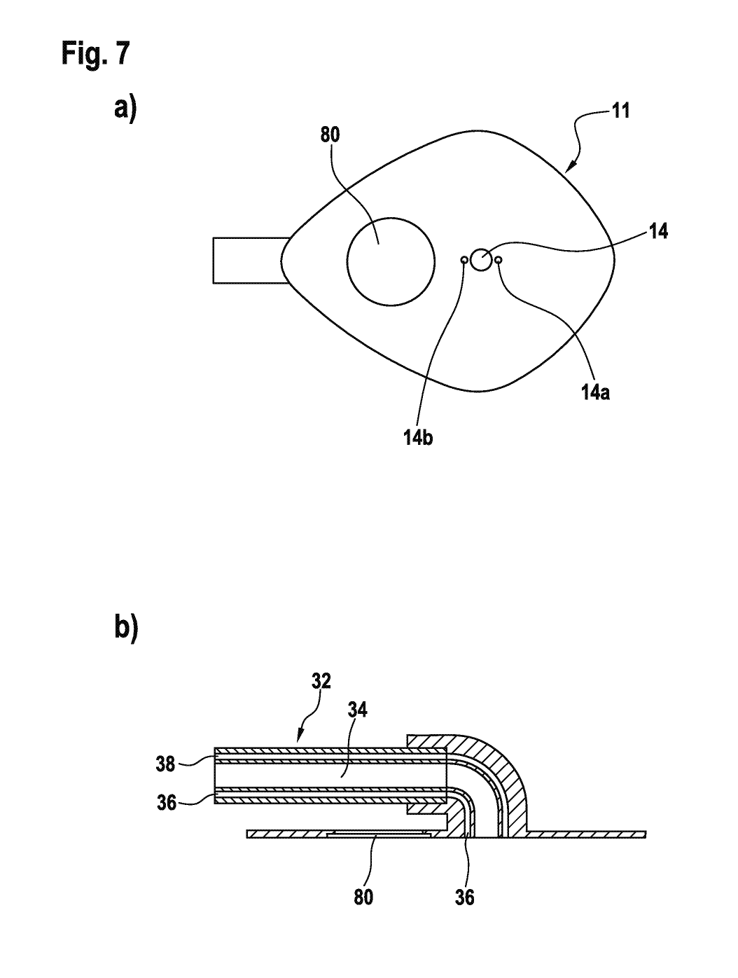

The suction connection according to FIG. 5 differs from the suction connection according to FIGS. 1 and 2 essentially in that the application area 11, in addition to a suction opening 14, also presents a ventilation opening 80 through which the wound can be ventilated in a controlled manner, to improve in that manner the flow conditions in the wound area, in terms of an improvement of the exudate removal. The ventilation opening 80 presents a larger diameter than the suction opening 14. As can be seen particularly clearly in the representation in FIG. 5b, the margin which surrounds the ventilation opening 80 in the application area 11 presents a ledge which can be used, according to FIGS. 6 and 7, as application surface for an antibacterial filter 82. As a result, the flow resistance in the area of the ventilation opening 80 is increased, which, however, can be compensated again by adjusting the diameter of the ventilation opening 80. In the context of the invention, the flow resistance of the filter 82 can be used to control the air flow. As a result, the flow resistance can be increased by decreasing the size of the filter pores. This control promotes the generation of a negative pressure with simultaneous ventilation. If the ventilation opening 80 is selected so it is excessively large, the negative pressure cannot be generated without using a filter. The ventilation opening 80 can also be arranged above the cover film 60 (in top view). It is essential that air is supplied to the wound. For this purpose, it may be advantageous to provide a slightly offset hole in the cover film 60, so that the air can flow without impediment to the ventilation opening 80.

The drainage space of the drainage layer 40 can be sufficient to allow air to flow into the central opening, even if the ventilation opening 80 is covered on the side facing the filler material 50 with the cover film 60.

In the embodiment represented in the drawing, the antibacterial filter 82 is made of polytetrafluoroethylene, and presents a pore size of approximately 0.001, particularly 0.005, preferably 0.02, particularly preferably 0.1-5 μm. In the wound treatment arrangement according to FIGS. 6a and 6b, it is covered with the drainage layer 40. As explained in reference to FIGS. 6a and 6b, the suction connection according to FIG. 5 as well as the suction connection according to FIG. 1 can be applied in accordance with the representations in FIGS. 3 and 4, so that it is either glued to a cover film 60 or overlapped by the cover film 60. It is essential that the ventilation opening 80, on the side facing away from the filler material 50, is not covered by the cover film 60. If the ventilation opening 80 or the filter 82 is covered by the cover film 60 on the side facing the filler material 50, the air can nevertheless flow into the wound area, through the space formed between two layers of the drainage film.

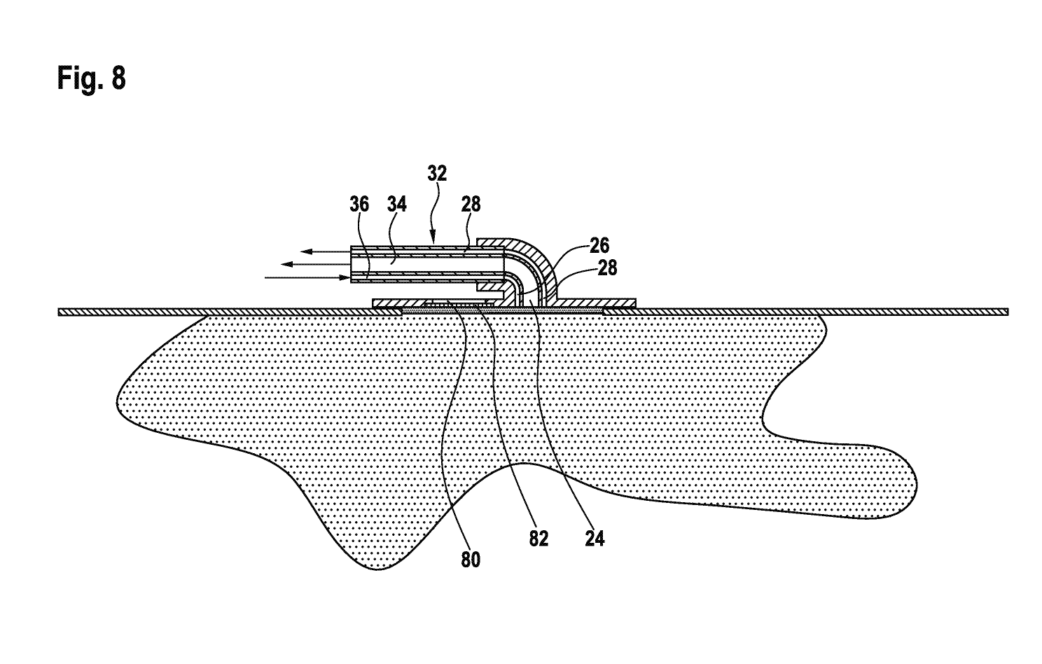

The embodiment according to FIG. 7 differs from the embodiment according to FIG. 5 essentially in that the suction tube is produced as a three-lumen tube 32, wherein, as diagrammatically indicated in FIG. 8, the middle and largest lumen 34 is used to generate the negative pressure in the wound area, a small lumen 36 can be used to ventilate the wound, and an additional, smaller lumen 38 is intended to measure the pressure in the wound area. The connection area 22 also presents the lumens 24, 26 and 28 corresponding to the lumens 32, 34 and 36.

The embodiment according to FIGS. 7 and 8 also presents, besides the ventilation lumen 26, a ventilation opening 80, to be able to further improve the exudate management.

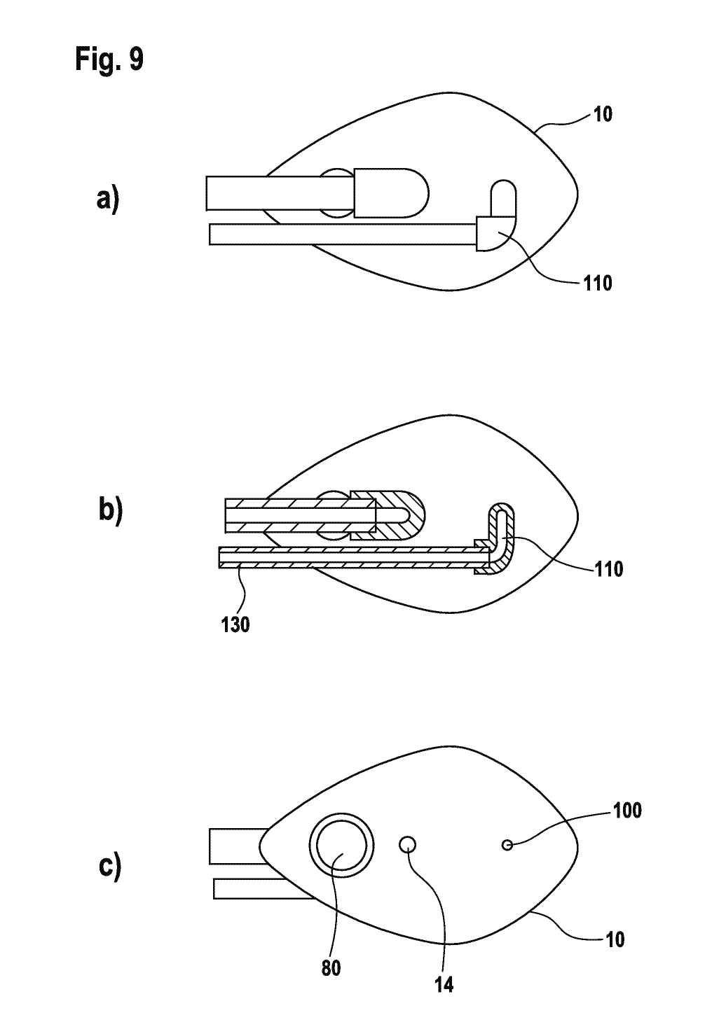

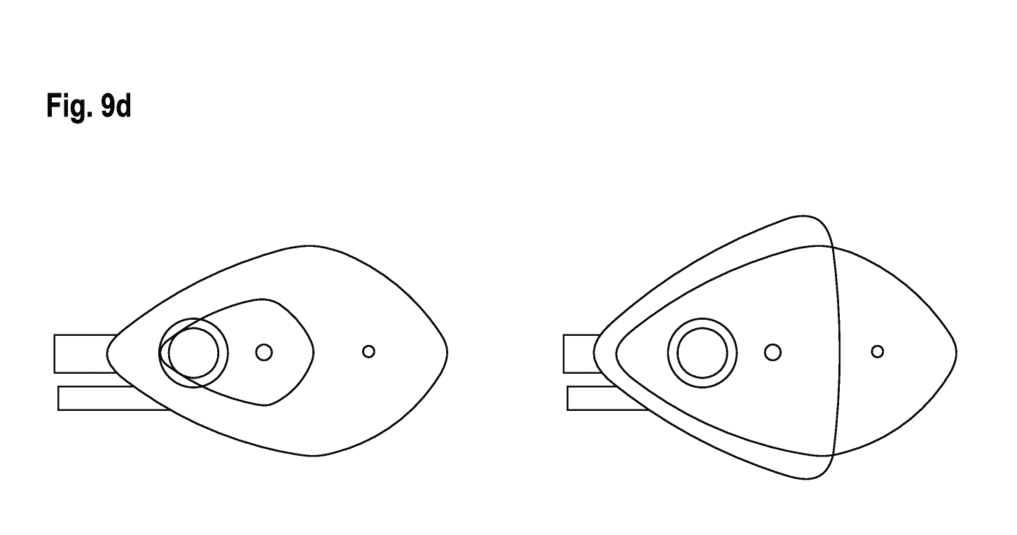

The embodiment according to FIG. 9 differs from the embodiment according to FIG. 5 essentially in that, in addition to the suction opening 14 and the ventilation opening 80, a supply opening 100 leading into the application area 11 of the suction connection 10 is also provided, which supply opening is associated with an additional pipe connector 110 for connecting the supply opening 100 to an additional tube 130. Via the tube 130, the additional pipe connectors 110 and the supply opening 100, wound treatment agents, such as, for example, a rinsing solution also mixed optionally with drugs, disinfectants or the like, can be introduced into the wound area. The embodiment according to FIG. 9 can also be used without using a separate ventilation opening 80, because the supply opening 100 can also be used for the ventilation. However, in terms of a good exudate management, it has been found to be particularly advantageous if both a ventilation opening 80 and also a supply opening 100, are provided next to the suction opening 14 in the application area 11 of the suction connection 10.

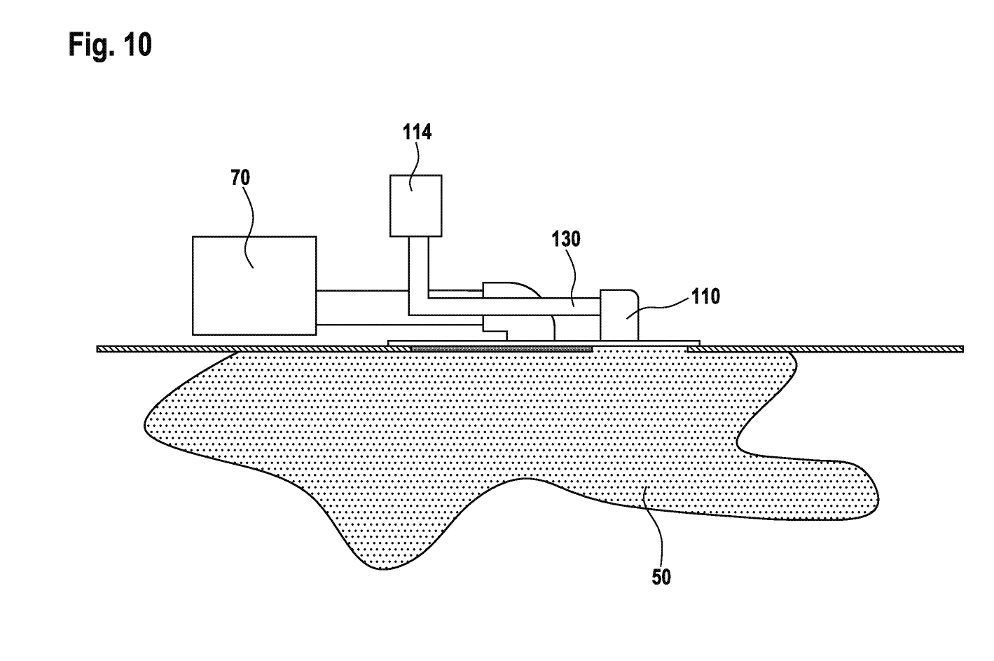

As one can see in FIG. 10, the rinsing solution can be introduced via the additional tube 130 and the additional pipe connector 110 with the help of a corresponding dosing element 114 into the area of the wound. Here, it has been found to be particularly advantageous if the supply opening 100 is not covered by the drainage layer 40, in order to ensure that the suctioning of wound exudate occurs without affecting the supplied rinsing solution, and in order to prevent that the supplied rinsing solution is immediately removed again by suction.

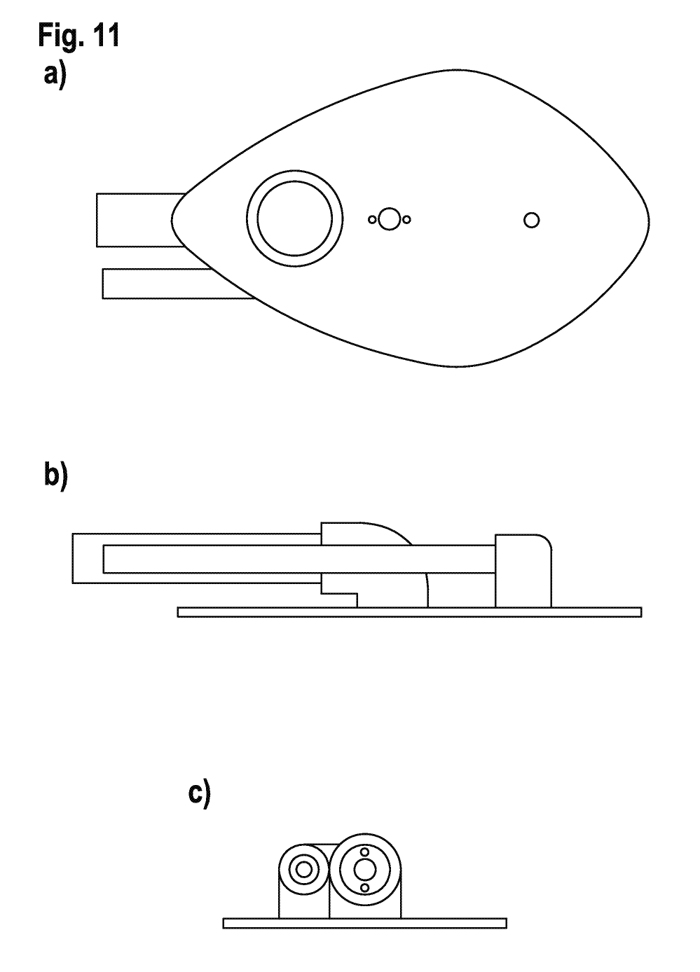

The embodiment according to FIG. 11 differs from the embodiment explained in reference to FIG. 9 in that a three-lumen tube 132 is used, the design and function of which are similar to the design according to FIG. 7. For this purpose, the design according to FIG. 11 presents a three-lumen tube to suction wound exudate and to ventilate the wound area, and, for the pressure measurement, it presents a ventilation opening 80 as well as a supply opening 100. As a result, an optimal wound management can be ensured.

The embodiments explained in reference to FIGS. 5-11, in contrast to the embodiments explained in reference to FIGS. 1-4, present an application area having a shape different from that of a circle, namely a rhombic shape. The corners of the rhombi are rounded. Due to the rhombic shape, a longitudinal axis is provided, which allows the arrangement of the suction opening, the ventilation opening and the supply opening on one line.

The invention is not limited to the embodiment examples explained in reference to the drawing. Rather, it also provides for the use of suction connections having a shape different from that of a circular disk or the shape of a rhombus, such as, for example, oval or rectangular suction connections.

Furthermore, the suction connection can also be slid onto the pipe connectors of the suction connection.

It is essential in the context of the invention that a drainage layer, for example of the type indicated in DE 10 2009 019 646 A1, is provided between the application surface of the suction connection and the filler material.

### LIST OF REFERENCE NUMBERS

10 Suction connection
11 Application area
12 Application surface
14 Suction opening
16, 16′ Attachment area
20 Connection device/pipe connector
22 Connection area
24 Lumen (negative pressure)
26 Ventilation lumen
28 Lumen (pressure measurement)
30 Suction tube
32 Three-lumen tube
34 Lumen (negative pressure)
36 Ventilation lumen
38 Lumen (pressure measurement)
40 Drainage layer
50 Filler material
60 Cover device/cover film
70 Suction device
72 Collecting vessel
80 Ventilation opening
82 Antibacterial filter
100 Supply opening
110 Pipe connector
114 Dosing element
130 Supply tube
132 Three-lumen tube

1. A wound treatment arrangement comprising:
a filler material designed to fill a wound,
a suction connection which is designed to suction wound exudate out of the filler material, and configured to be arranged on the side facing away from a wound bed of the wound, outside of the filler material, and
a drainage layer arranged between the filler material and the suction connection for introducing the exudate to be suctioned out of the filler material into at least one suction opening of the suction connection.

2. The wound treatment arrangement of claim 1, wherein the suction opening penetrates through a flange-like application area having a substantially flat application surface of the suction connection, configured to be applied on the drainage layer.

3. The wound treatment arrangement of claim 2, wherein on the side of the suction connection, which faces away from the application surface, a connection device is provided for establishing a connection between the suction opening and a suction tube.

4. The wound treatment arrangement of claim 2, wherein the drainage layer is attached to the application surface, particularly glued, welded to the application surface, clamped and/or sewn on the application surface.

5. The wound treatment arrangement claim 2, characterized in that the suction connection, particularly in the flange-like application area, is penetrated by a ventilation opening provided for the ventilation of the wound.

6. The wound treatment arrangement of claim 5, wherein the ventilation opening is associated with an antibacterial filter.

7. The wound treatment arrangement claim 3, wherein the suction tube is a multi-lumen tube, particularly a tube with three lumens.

8. The wound treatment arrangement of claim 2, further comprising a supply opening configured to supply a wound treatment product and penetrates through the application area.

9. The wound treatment arrangement of claim 8, wherein on the side of the suction connection, which faces away from the application surface, an additional connection device is arranged for establishing a connection between the supply opening and a supply tube.

10. The wound treatment arrangement of claim 1, further comprising an additional contact layer arranged between the wound bed and the filler material.

11. The wound treatment arrangement of claim 1, wherein the drainage layer and/or the contact layer comprises two approximately mutually parallel track-shaped elements, between which a drainage space is formed, the depth of which ensures, in a depth direction extending perpendicularly to the track-shaped elements, a capillary action on the exudates received in the drainage space.

12. The wound treatment arrangement of claim 11, wherein the depth of the drainage space is 5 mm or less.

13. The wound treatment arrangement of claim 1, further comprising a cover device, which can be fixed in an air-tight manner to skin surrounding the wound, and which is provided to form a space which is closed off in an air-tight manner and which contains the filler material.

14. The wound treatment arrangement of claim 2, wherein the drainage layer covers the application surface only partially, and wherein the wound treatment arrangement further comprises an attachment area of the application surface, which surrounds the drainage layer and can be glued to the cover device.

15. The wound treatment arrangement of claim 13, wherein the drainage layer completely covers the application surface, and the cover device is glued to an attachment area of the suction connection, which faces away from the application surface, and surrounds the connection device.

16. The wound treatment arrangement of claim 1, wherein the filler material comprises an open-pored foam and/or gauze.

17. A suction connection for a wound treatment arrangement which is designed to suction wound exudate out of a filler material of a wound treatment arrangement, and configured to be arranged on a side of the wound treatment arrangement facing away from a wound bed and outside of a filler material of the wound treatment arrangement.

18. The suction connection of claim 17, further comprising a drainage layer attached to an application surface of the suction connection.

19. The suction connection of claim 17, further comprising a flange-like application area which is penetrated by a suction opening, wherein an application area is, completely perforated by a ventilation opening and/or a supply opening which is provided to supply a wound treatment product.

20. The wound treatment arrangement of claim 3, wherein the connection device comprises a pipe connector.

21. The wound treatment arrangement of claim 4, wherein the drainage layer is glued, welded, clamped, or sewn to the application surface.

22. The wound treatment arrangement of claim 9, wherein the additional connection device comprises an additional pipe connector.

23. The wound treatment arrangement of claim 12, wherein the depth of the drainage space is, and at least 0.5 mm.

24. The wound treatment arrangement of claim 13, wherein the cover device comprises a cover film.

25. The suction connection of claim 18, wherein the drainage layer is glued, welded, clamped, or sewn to the application surface.
